(19) United States
(12) Patent Application Publication
(10) Pub. No.: US 2011/0262939 A1
(43) Pub. Date: Oct. 27, 2011

(54) METHODS AND ASSAYS FOR CLASSIFYING FOODSTUFF AND/OR BEVERAGE AND/OR DIET AND/OR NUTRITION REGIMEN AND/OR MEDICAMENT IN VIEW OF AN EFFECT ON THE CARDIOVASCULAR SYSTEM

(75) Inventors: Andreas Bergmann, Berlin (DE); Andreas Pfeiffer, Nuthetal (DE); Natalia Rudovich, Nuthetal (DE)

(73) Assignee: B.R.A.H.M.S GmbH, Hennigsdorf (DE)

(21) Appl. No.: 13/126,623

(22) PCT Filed: Oct. 29, 2009
(86) PCT No.: PCT/EP2009/007924

§ 371 Date: Jul. 18, 2011

(30) Foreign Application Priority Data
Oct. 31, 2008 (EP) 08168107.4

(51) Int. Cl. G01N 33/566; G01N 33/53

(52) U.S. Cl. 435/7.92; 436/501

(57) ABSTRACT

Subject of the present invention is an in vitro-method for classifying a foodstuff and/or beverage and/or diet and/or nutrition regimen and/or medicament in view of an effect on the cardiovascular system of a subject, comprising determining the relative level of one or more cardiovascular markers.

Subject of the invention is an in vitro-method for classifying a foodstuff and/or beverage and/or diet and/or nutrition regimen and/or medicament in view of an effect on the cardiovascular system of a subject, comprising determining the relative level of one or more cardiovascular markers as well as uses thereof.

Hypertension frequently accompanies obesity, hyperinsulinism and insulin resistance. This type of hypertension is characterized by sodium retention, an increased intravascular volume and increased cardiac stroke volume and output (Messerli F H, et al. 1981 Obesity and essential hypertension. Hemodynamics, intravascular volume, sodium excretion, and plasma renin activity. Arch Intern Med 141:81-5; Stelfox H T, et al. 2006 Hemodynamic monitoring in obese patients: the impact of body mass index on cardiac output and stroke volume. Crit Care Med 34:1243-6). Atrial natriuretic peptide (ANP) and Brain type natriuretic peptide (BNP) are synthesized in myocardial cells as a response to increased wall stress in relation to heart failure or acute myocardial ischemia as prohormones that are cleaved into ANP and BNP and N-terminal proANP (NT-proANP) as well as N-terminal proBNP (NT-proBNP), respectively (Ruskoaho H 2003 Cardiac hormones as diagnostic tools in heart failure. Endocr Rev 24:341-56; Potter L R, et al. 2006 Natriuretic peptides, their receptors, and cyclic guanosine monophosphate-dependent signaling functions. Endocr Rev 27:47-72). High levels of natriuretic peptides are new promising cardiovascular (CV) risk markers and have been associated with high blood pressure (BP), left ventricular hypertrophy, and albuminuria (Olsen M H, et al. 2005 N-terminal pro brain natriuretic peptide is inversely related to metabolic cardiovascular risk factors and the metabolic syndrome. Hypertension 46:660-6). Several large studies indeed observed independent associations of mortality and natriuretic peptide levels (Wang T J, et al. 2004 Plasma natriuretic peptide levels and the risk of cardiovascular events and death. N Engl J Med 350:655-63; Bibbins-Domingo K, et al. 2007 N-terminal fragment of the prohormone brain-type natriuretic peptide (NT-proBNP), cardiovascular events, and mortality in patients with stable coronary heart disease. Jama 297:169-76; Kistorp C, et al. 2005 N-terminal pro-brain natriuretic peptide, C-reactive protein, and urinary albumin levels as predictors of mortality and cardiovascular events in older adults. Jama 293:1609-16; von Haehling, et al. 2007 Comparison of midregional pro-atrial natriuretic peptide with N-terminal pro-B-type natriuretic peptide in predicting survival in patients with chronic heart failure. J Am Coll Cardiol 50:1973-80).

Recent studies have described that natriuretic peptide levels are suppressed in obesity (Wang T J, et al. 2004 Impact of obesity on plasma natriuretic peptide levels. Circulation 109:594-600; Das S R, et al. 2005 Impact of body mass and body composition on circulating levels of natriuretic peptides: results from the Dallas Heart Study. Circulation 112:2163-8). Since obesity is associated with salt retention and increased cardiac output it would be expected to produce elevated natriuretic peptide levels. That obesity seemed to have the opposite effect appeared counterintuitive and was attributed to nonhemodynamic factors. Wang and colleagues therefore postulated that this inverse relationship may be due to increased expression of the natriuretic peptide clearance receptor (NPR-C) by adipose tissue resulting in increased clearance of natriuretic peptides in obese subjects (Wang T J, et al. 2004 Impact of obesity on plasma natriuretic peptide levels. Circulation 109:594-600). However, Das and colleagues determined lean and fat mass by DEXA in the Dallas heart study and observed an association of lower BNP levels with lean rather than fat mass (Das S R, et al. 2005 Impact of body mass and body composition on circulating levels of natriuretic peptides: results from the Dallas Heart Study. Circulation 112:2163-8).

Several studies observed an association of natriuretic peptides with further components of the metabolic syndrome (Olsen M H, et al. 2008 Cardiovascular risk prediction by N-terminal pro brain natriuretic peptide and high sensitivity C-reactive protein is affected by age and sex. J Hypertens 26:26-34; Wang T J, et al. 2007 Association of plasma natriuretic peptide levels with metabolic risk factors in ambulatory individuals. Circulation 115:1345-53). Elevated waist circumference, elevated triglycerides, reduced HDL, and elevated fasting glucose (Wang T J, et al. 2007 Association of plasma natriuretic peptide levels with metabolic risk factors in ambulatory individuals. Circulation 115:1345-53) were associated with lower plasma ANP levels, and somewhat less with lower BNP levels in the Framingham heart study. In a Danish study, an association of BNP with BMI, insulin, glucose, triglycerides and hypertension was observed (Olsen M H, et al. 2005 N-terminal pro brain natriuretic peptide is inversely related to metabolic cardiovascular risk factors and the metabolic syndrome. Hypertension 46:660-6). Although these studies demonstrated close links of the natriuretic peptides to several traits of the metabolic syndrome, the mechanisms behind these associations have remained elusive.

Nutritional approaches reducing postprandial insulin levels are highly effective in reducing blood pressure (Appel L J, et al. 2005 Effects of protein, monounsaturated fat, and carbohydrate intake on blood pressure and serum lipids: results of the OmniHeart randomized trial. Jama 294:2455-64) and the postprandial state is believed to play an important role in the early stages of the metabolic syndrome and particularly in the development of atherosclerosis (Hanefeld M, et al. 1999 Postprandial plasma glucose is an independent risk factor for increased carotid intima-media thickness in non-diabetic individuals. Atherosclerosis 144:229-35; Hanefeld M, et al. 2007 The challenge of the Metabolic Syndrome. Horm Metab Res 39:625-6).

Object of the present invention was the classification of a foodstuff and/or beverage and/or diet and/or nutrition regimen and/or medicament in view of an effect on the cardiovascular system of a subject.

Surprisingly, it was found that insulin induces suppression of ANP. This connection provides a direct link from insulin resistance to hypertension in the metabolic syndrome. The finding of this connection further lead to methods for classifying a foodstuff and/or beverage and/or diet and/or nutrition regimen and/or medicament in view of an effect on the cardiovascular system of a subject and uses thereof.

Thus, subject of the present invention is an in vitro-method for classifying a foodstuff and/or beverage and/or diet and/or nutrition regimen and/or medicament in view of an effect on the cardiovascular system of a subject, comprising determining the relative level of one or more cardiovascular markers in said subject.

According to the present invention “relative level” is defined as being the relative concentration based on a basal value, which can be mathematically expressed as follows:

$$X = 100 \times \frac{[ postprandial ]}{[ basal ]}$$

wherein X is the change of the level of one or more cardiovascular markers in percent.

It can also be specified as percentaged change of the concentration relative to the basal value, which can be mathematically expressed as follows:

$$X = 100 - \frac{[ postprandial ]}{[ basal ]} * 100 or X = \frac{[ postprandial ]}{[ basal ]} * 100 - 100$$

In a preferred embodiment of the inventive method the postprandial relative level of one or more cardiovascular markers is determined.

In the context of the present invention the term “postprandial” refers to the period of time after the foodstuff and/or beverage and/or medicament is ingested by or applied otherwise to the subject, which may also be in the context of a diet and/or nutrition regimen.

In the context of the present invention cardiovascular marker means a peptide and/or protein providing diagnosis and/or prognosis and/or monitoring of cardiovascular diseases (e.g. myocardial infarction, coronary artery disease, heart failure) selected from the group of natriuretic peptides (e.g. atrial natriuretic peptide, brain natriuretic peptide), adrenomedullin, endothelins, vasopressin. The basal level of the cardiovascular markers depends on such factors as the subject's age, body mass index, genetic predisposition for certain conditions/family history, gender and ethnic background of the patient, as well as on the overall health status of said subject. The present invention, however, is based on the finding that in contrast to this, the relative change from the basal level of the cardiovascular markers to the postprandial level of the cardiovascular markers is essentially independent from these factors and strongly depends on foodstuff and/or beverage and/or diet and/or nutrition regimen and/or medicament which is administered to the subject.

In an especially preferred embodiment of the inventive method the relative level of one or more cardiovascular markers is determined with an assay having a lower detection limit of 1 nmol/L or lower, preferably 100 pmol/L or lower, more preferably 10 pmol/L or lower, even more preferably 1 pmol/L or lower, most preferably 0.5 pmol/L or lower. Furthermore, the assay preferably has an interassay precision of <30%, more preferably <20% in the normal range. Furthermore, the assay preferably has an intraassay precision of <10%, more preferably <5% in the measuring range. Hereby “intraassay precision” specifies the deviance between measurements within a single batch of a specific assay, and “interassay precision” specifies the deviance between measurements within multiple batches of a specific assay, which may be carried out in different locations, on different days, by different operators. Thus, the aforementioned terms are related to a measure of the reproducibility of results obtained with the concerned assays. “Measuring range” specifies the upper and lower limit of detection of an assay.

The assay is at least sensitive enough to detect changes and variances as increase and as decrease. For a healthy subject the normal range of a given biomarker corresponds to a Gaussian distribution.

An embodiment of the invention is further an in vitro-method according to the present invention, further comprising:

a) determining the basal level of one or more cardiovascular markers in said subject,

b) determining the postprandial level of said one or more cardiovascular markers,

c) calculating the relative level of one or more cardiovascular markers from the values obtained in steps a and b.

Hereby, the ingestion, intake or other form of application of said foodstuff and/or beverage and/or diet and/or nutrition regimen and/or medicament is correlated to its influence on the level of said one or more cardiovascular markers in said subject in terms of a relative decrease or increase of said level.

In the context of the present invention, the term “basal level” refers to the individual level of a certain compound, molecule or metabolite, such as a cardiovascular peptide, which a subject has without the influence of factors such as a foodstuff, a beverage, a diet, a nutrition regimen or a medicament. Said basal level is individually determined for each subject after approximately 12 hours of fasting. Fasting hereby means that the subject does not ingest or otherwise consume foodstuffs, beverages or medicaments for a certain amount of time, except water and/or indispensable medication.

In a preferred embodiment of the invention a postprandial relative increase of the level of said one or more cardiovascular markers of more than 5%, preferably between more than 5 and up to 20% in said subject is correlated with a positive effect of said foodstuff and/or beverage and/or diet and/or nutrition regimen and/or medicament on the cardiovascular system of said subject, wherein a postprandial relative increase or decrease of the level of said one or more cardiovascular markers of about 5% in said subject is correlated to a neutral effect of said foodstuff and/or beverage and/or diet and/or nutrition regimen and/or medicament on the cardiovascular system of said subject, and wherein a postprandial relative decrease of the level of said one or more cardiovascular markers of more than 5% in said subject is correlated to a negative effect of said foodstuff and/or beverage and/or diet and/or nutrition regimen and/or medicament on the cardiovascular system of said subject.

A positive effect on the cardiovascular system is, in a medical sense, the improvement of the cardiovascular system of said subject, or, if the subject is healthy, the preservation of the medical condition of said subject.

A neutral effect on the cardiovascular system is, in a medical sense, the preservation of the medical condition of the cardiovascular system of said subject.

A negative effect on the cardiovascular system is, in a medical sense, the pejoration of the medical condition of the cardiovascular system of said subject.

A positive effect on the cardiovascular system of said subject may include cardiovascular remodelling and improvement of cardiovascular function, which may especially and preferably include the prevention of atherosclerosis, hypertension and cardiac and vascular dysfunction and development of heart failure.

In another preferred embodiment the basal level of said cardiovascular marker and the post-prandial level of said cardiovascular marker in said subject are determined with an immunoassay.

The diagnostic assay can be of any type applied in the field of diagnostics, including but not restricted to assay methods based on enzymatic reactions, luminescence, fluorescence, or radiochemicals. The preferred detection methods comprise strip tests, radioimmunoassay, chemiluminescence- and fluorescence-immunoassay, Immunoblot assay, Enzyme-linked immunoassay (ELISA), Luminex-based bead arrays, and protein microarray assay. The assay types can further be microtiter plate-based, chip-based, bead-based, wherein the biomarker proteins can be attached to the surface or are in solution. The assays can be homogenous or heterogeneous assays, sandwich assays, competitive and non-competive assays (The Immunoassay Handbook, Ed. David Wild, Elsevier LTD, Oxford; 3rd ed. (May 2005), ISBN-13: 978-0080445267; Hultschig C et al., Curr Opin Chem Biol. 2006 February;10(1):4-10. PMID: 16376134).

In the most preferred embodiment of the invention an immunoassay is used as described in (Morgenthaler N G et al; 2004 ClinChem 50:234-6).

In an especially preferred embodiment of the in vitro-method according to the invention one of the cardiovascular markers is proANP. It is even more preferred that one of the markers is midregional proANP. Mostly preferred is midregional proANP₅₃₋₉₀.

In another preferred embodiment of the in vitro-method according to the invention the post-prandial level of said one or more cardiovascular markers is determined within 4 hours, preferably within 2 hours, more preferably between 15 and 60 minutes after administration of said foodstuff and/or beverage and/or diet and/or nutrition regimen.

In another preferred embodiment of the in vitro-method according to the invention the post-prandial influence of said foodstuff and/or beverage and/or diet and/or nutrition regimen on the relative level of one or more cardiovascular peptides is monitored over a prolonged period, preferred over a period of one week, more preferred one month, even more preferred two months, even more preferred half a year.

Subject of the invention is further the use of an assay, preferably having a sensitivity of 1 nmol/L or lower, preferably 100 pmol/L or lower, more preferably 10 pmol/L or lower, even more preferably 1 pmol/L or lower, most preferably 0.5 pmol/L or lower for the methods of the present invention.

In one aspect of the method according to the invention the change of the level of one or more cardiovascular markers of a subject relative to the basal level of said markers of said subject is determined, wherein the change is a decrease.

Subject of the invention is further the use of an assay for determining the change of the level of one or more cardiovascular markers of a subject relative to the basal level of said markers of said subject, wherein the assay is capable of detecting a decrease of the level of said one or more cardiovascular markers and capable of detecting an increase of the level of said one or more cardiovascular markers.

It is important to note that the capability of the assay used in the present invention to measure a decrease in the level of said one or more cardiovascular markers is critical, in particular in healthy subjects, wherein the decrease leads to very low levels of said one or more cardiovascular markers. Thus, the assay used herein preferably is ultra-sensitive in order to be capable of measuring a decrease in the level of said one or more cardiovascular markers, in subjects in which the basal level lies within the 97.5^{th} percentile of said level in the healthy population.

Subject of the invention is further the use of an assay as described above, wherein the change is an increase or a decrease, and wherein the assay has sensitivity of 1 nmol/L or lower.

Subject of the invention is further the use of an assay as described above, for determining the postprandial change of the level of one or more cardiovascular markers of a subject relative to the basal level of said markers of said subject.

Subject of the invention is further the use of an assay having a sensitivity of 1 nmol/L or lower, preferably 100 pmol/L or lower, more preferably 10 pmol/L or lower, even more preferably 1 pmol/L or lower, most preferably 0.5 pmol/L or lower for determining the change of the level of one or more cardiovascular markers of a subject relative to the basal level of said markers of said subject, wherein the change is an increase or a decrease. In a preferred embodiment of the inventive method the postprandial relative level of one or more cardiovascular markers is determined In an especially preferred embodiment one of the cardiovascular markers is proANP. It is even more preferred that one of the markers is mid-regional proANP. Mostly preferred is midregional proANP₅₃₋₉₀.

MR-proANP₅₃₋₉₀ specifies the midregional pro-atrial natriuretic peptide, which comprises amino acids 53 to 90 of the pro-atrial natriuretic peptide (proANP), FIG. 1.

Subject of the invention is further the use of an assay, preferably having a sensitivity of 1 nmol/L or lower, preferably 100 pmol/L or lower, more preferably 10 pmol/L or lower, even more preferably 1 pmol/L or lower, most preferably 0.5 pmol/L or lower, for determining the to postprandial change of the level of one or more cardiovascular markers of a subject relative to the basal level of said markers of said subject, wherein the change is an increase or a decrease.

It is preferred that the assay is an immunoassay.

Subject of the invention is further the use of an assay as outlined above, wherein said change in the level of one or more cardiovascular markers is used to classify a foodstuff and/or beverage and/or diet and/or nutrition regimen and/or medicament in view of an effect on the cardiovascular system of a subject.

In a particular embodiment said change in the level of one or more cardiovascular markers is used to classify a diabetes medicament.

In one embodiment of the invention said subject is a healthy or non-symptomatic human being.

In another embodiment of the invention said subject is a human being having a medical condition associated with the cardiovascular system and/or metabolic syndrome.

As used herein, the term “metabolic syndrome” refers to the aggregation of several risk factors for cardiovascular diseases and type II diabetes, as defined by the American Heart Association (AHA) and National Heart, Lung and Blood Institute (NHLBI) (Grundy et al. 2005. Circulation 112: 2735-3752), incorporated herein by reference. Important components of the metabolic syndrome are, among others, glucose intolerance and dyslipidemias, hypertension and central obesity. The metabolic syndrome is diagnosed if 3 of the following 5 criteria are fulfilled: elevated waist circumference (≧102 cm in male and ≧88 cm in female), elevated triglycerides (≧150 mg/dL/ 1.7 mmol/L, respectively, or drug treatment for elevated triglycerides), reduced HDL-cholesterol (<40 mg/dL/ 1.03 mmol/L, respectively, in male; <50 mg/dL/ 1.3 mmol/L, respectively, in female; or drug treatment for reduced HDL-cholesterol), elevated blood pressure (≧130 mm Hg systolic blood pressure or ≧85 mm Hg diastolic blood pressure or on antihypertensive drug treatment in a subject with a history of hypertension) and elevated fasting glucose (≧100 mg/dL or drug treatment for elevated glucose).

A more recent definition with some modifications has been given by the International Diabetes Federation (http://www.idf.org/webdata/docs/IDF_Meta_def_final.pdf).

In one embodiment of the invention said subject is a human being suffering from diabetes, in particular type II diabetes.

In a preferred embodiment of the invention, the condition associated with the cardiovascular system and/or metabolic system is the metabolic syndrome.

A condition associated with the cardiovascular system and/or metabolic system may also comprise a state after incidents of the cardiovascular system and/or metabolic system. In these cases healthy nutrition may improve the subject's chance of survival (de Lorgeril M, et al. Mediterranean diet, traditional risk factors, and the rate of cardiovascular complications after myocardial infarction: final report of the Lyon Diet Heart Study. Circulation 1999; 99(6): 779-85).

In another preferred embodiment of the invention, the condition associated with the cardiovascular system and/or metabolic system is selected from the group comprising myocardial infarction (MI), coronary syndromes, congestive heart failure (CHF), coronary artery disease (atherosclerosis), stroke, transient ischemic attacks (TIA), periphery artery disease, cardiomyopathy, diabetes mellitus type II, renal failure and/or subjects with one or more symptoms of the above mentioned diseases, e.g. obesity, hypertension, headache, chest pain and dyspnea.

According to the present invention it is preferred that at least one of the markers is a peptide selected from the group comprising natriuretic peptides, endothelin-1 (ET-1), vasopressin (AVP), adrenomedullin (ADM), as well as propeptides thereof and fragments of at least 3 amino acids thereof, preferably more than 5, more preferably more than 6, even more preferably more than 7, even more preferably more than 10, even more preferably more than 12, even more preferably more than 15, most preferably 20 or more.

Natriuretic peptide refers to a peptide which induces natriuresis (the discharge of sodium through urine). Types include:
Artrial natriuretic peptide,
Brain natriuretic peptide,
C-type natriuretic peptide.

According to the present invention said marker is a natriuretic peptide or a propeptide or fragments of at least 3 amino acids thereof, preferably more than 5, more preferably more than 6, most preferably more than 7.

In a very preferred embodiment of the invention at least one of the cardiovascular markers is atrial natriuretic peptide (ANP) or a propeptide or fragments of at least 3 amino acids thereof, preferably more than 5, more preferably more than 6, most preferably more than 7.

In an especially preferred embodiment at least one of the cardiovascular markers is MR-proANP₅₃₋₉₀ or fragments of at least 3 amino acids thereof, preferably more than 5, more preferably more than 6, most preferably more than 7.

AVP in the context of the present invention relates to arginine vasopressin (=vasopressin) or fragments thereof or precursors or fragments thereof. A preferred fragment of a precursor of AVP is C-terminal proAVP (CT-proAVP or Copeptin). CT-proAVP₁₀₇₋₁₄₅ (or CT-pre-proAVP₁₂₆₋₁₆₄) is a particularly preferred marker peptide in the context of the present invention.

ADM in the context of the present invention relates to adrenomedullin or fragments thereof or precursors or fragments thereof. A preferred fragment of a precursor of ADM is mid-regional proADM (MR-proADM). MR-proADM₂₄₋₇₁ (or MR-preproADM₄₅₋₉₂) is a particularly preferred marker peptide in the context of the present invention.

ET-1 in the context of the present invention relates to endothelin 1 or fragments thereof or precursors or fragments thereof. A preferred fragment of a precursor of ET-1 is C-terminal-proET1 (CT-proET1). CT-proET-1₁₅₁₋₁₉₅ (or _{CT-preproET-1}₁₆₈₋₂₁₂) is a particularly preferred marker peptide in the context of the present invention.

### FIGURE DESCRIPTION

FIG. 1

Sequence of pro-ANP

FIG. 2

Sequence of pre-pro-ADM

FIG. 3

Sequence of pro-ADM

FIG. 4

Sequence of MR-pro-ADM

FIG. 5

Sequence of ADM

FIG. 6

Sequence of pre-pro-ET-1

FIG. 7

Sequence of pro-ET-1

FIG. 8

Sequence of ET-1

FIG. 9

Sequence of CT-pro-ET-1

FIG. 10

Sequence of Big-ET-1

FIG. 11

Sequence of pre-pro-AVP

FIG. 12

Sequence of pro-AVP

FIG. 13

Sequence of AVP

FIG. 14

Sequence of CT-pre-proAVP (Copeptin)

FIG. 15

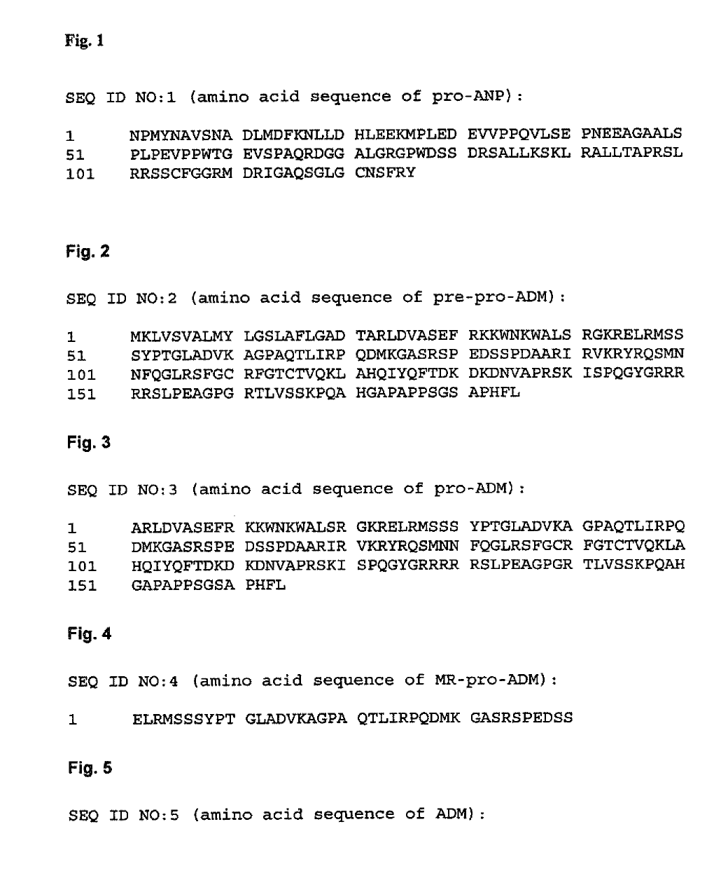

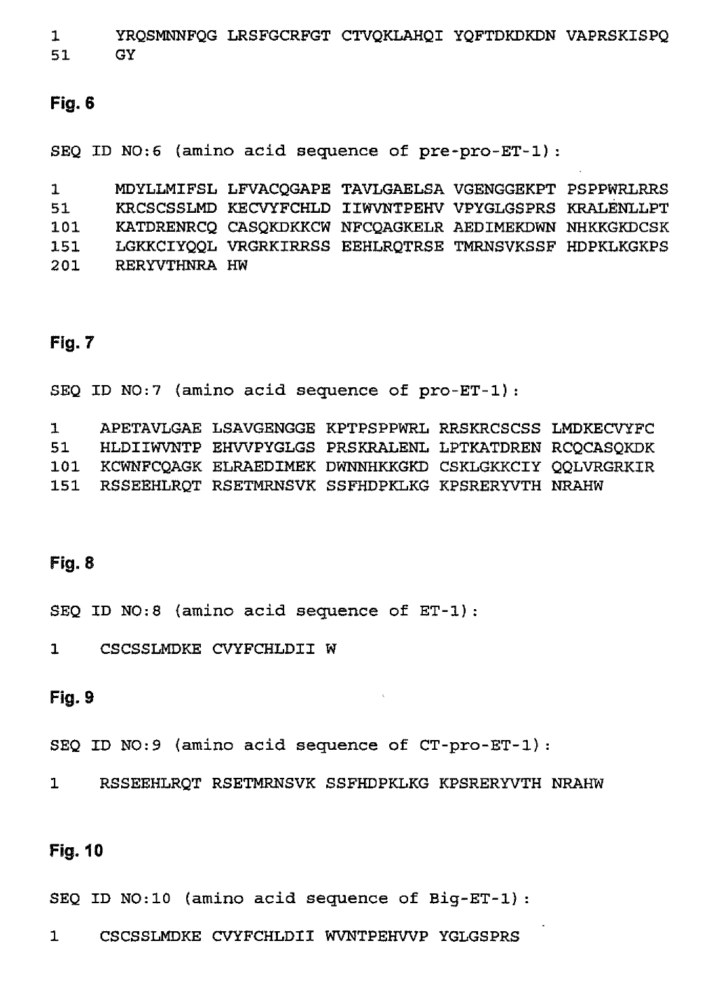

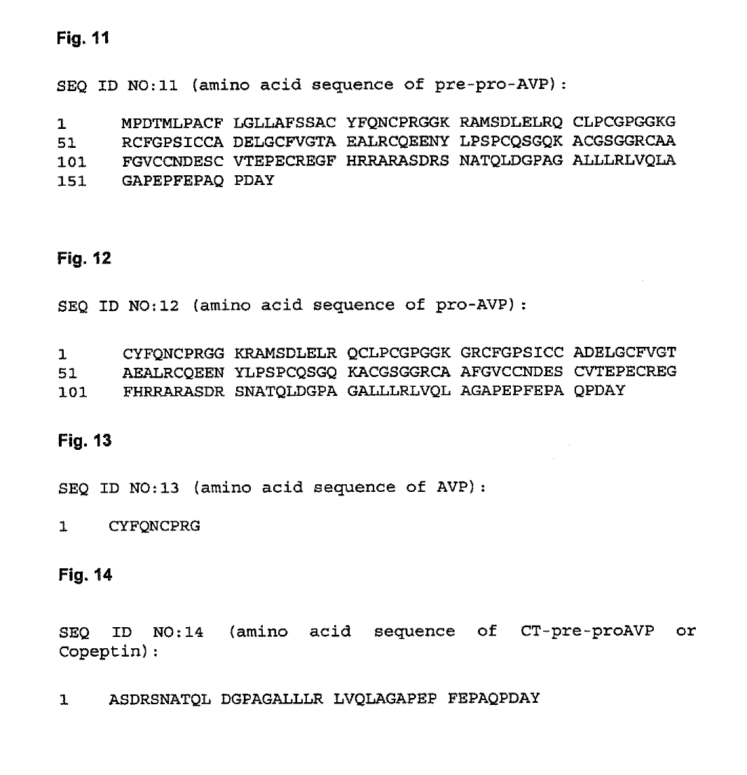

Plasma MR-proANP₅₃₋₉₀ (A) and serum insulin (B) during the oral glucose tolerance test in non obese normotensive subjects (black diamonds), normotensive subjects with central obesity (white diamonds) and hypertensive subjects (black triangles); *p<0.05 non obese vs. obese, normotensive subjects; †p<0.001 obese normotensive vs. hypertensive subjects; ‡p<0.01 hypertensive vs. non obese normotensive subjects.

(C) Supression of plasma MR-proANP₅₃₋₉₀ levels in the hyperinsulinemic, euglycemic clamps. (D) Delta MR-proANP_{0-120 min} in subjects with low and high insulin sensitivity, determined as glucose infusion rate (GIR) values in the steady-state of the clamp below the 25^{th} and above the 75^{th} percentile, respectively. Data are shown by box-and-whiskers-plots. The box extends from the 25^{th} to the 75^{th} percentile, with a line at the median indicating the 50^{th} percentile. The whiskers represent the ranges extending from the lowest to the highest value.

FIG. 16

Comparison of relative concentrations of NT-proBNP and MR-proANP₅₃₋₉₀ after oGTT in n=10 subjects.

### EXAMPLES

### Research Design and Methods

### Study Protocol

The study protocol was approved by the ethical committees of the Potsdam University and Charite University of Medicine, Berlin, Germany. Before the study, informed written consent was obtained from all participants.

### Study Design

The subjects are part of an ongoing case-control association study of the aetiology of the metabolic syndrome and type 2 diabetes mellitus (Metabolic Syndrome Berlin-Potsdam Study, MESY-BEPO). In Potsdam and Berlin, Germany, volunteers from the general population were recruited. The baseline examination included anthropometric measurements, blood sampling, a 75 g oral glucose tolerance test (oGTT) and personal interview on lifestyle habits and medical history. A subgroup of this population (n=31) underwent hyperinsulinemic, euglycemic clamps, which was conducted on a separate day after the oGTT.

### Subjects

One hundred and eight non hypertensive subjects (55 non-obese and 53 with central obesity) and 54 patients with an essential hypertension were studied. Hypertension was defined as systolic blood pressure ≧140 mm Hg, diastolic blood pressure ≧90 mm Hg, or use of antihypertensive therapy. All drug treated hypertensive subjects had a stable medication in the last six month prior the study. Subjects with elevations in liver enzymes more than twice the respective upper normal limits, or with elevated serum creatinine concentrations (>1.3 mg/dl) or with severe conditions including generalized inflammation, heart failure or end-stage malignant diseases were excluded from the study. All subjects were instructed to maintain their normal physical activity and to consume a normal diet containing ≧200 g of carbohydrate during three days before oGTT and clamp test. Subjects with antidiabetic therapy or newly diagnosed type 2 diabetes mellitus were excluded from the examination. Definitions of disturbances in the glucose metabolism were based on the 1997 American Diabetes Association criteria for glucose values obtained after an overnight fast and a two-hour 75 g oGTT (2000 Report of the Expert Committee on the Diagnosis and Classification of Diabetes Mellitus. Diabetes Care 23 Suppl 1:S4-19).

### Experimental Procedures

All tests were performed in the morning after 12 hours overnight fast. BP was measured by a trained study nurse using an Omron® HEM705CP manometer (Omron, Germany) with patients in the sitting position. Three measurements were taken at 2-min intervals and the average was used to define clinical systolic and diastolic blood pressures. For oGTT venous blood samples were drawn at 0, 30, 60, 90, 120 and 180 min relative to the oral glucose loading. Euglycemic, hyperinsulinemic clamp:

Hyperinsulinemic euglycemic clamps were performed for 120 min using 100 mU of human insulin per m² of the body surface per min (Actrapid; Novo Nordisk, Bagsværd, Denmark) and a variable infusion of 20% glucose (Serag Wiessner, Naila, Germany) (DeFronzo R A, et al. 1979 Glucose clamp technique: a method for quantifying insulin secretion and resistance. Am J Physiol 237:E214-23). In the steady-state condition of the clamp, capillary blood glucose was adjusted at 5.5 mmol/l for at least 60 min A deviation of a single capillary glucose concentration of >10% during assumed steady-state conditions was defined as non-steady state. Throughout the clamp, capillary blood glucose concentrations were monitored every 5 min and used to regulate plasma glucose by the adjustment of a variable infusion of glucose.

### Analytical Procedures

All venous blood samples were immediately centrifuged and frozen at −70° C. until analyzed. Capillary blood glucose concentrations were determined using a glucose oxidase method on Dr. Mëdler G-L (Dr. Mëller Glucose analyzer, Freital, Germany). Serum triglycerides, total cholesterol and HDL-cholesterol were determined by standard enzymatic assays, and LDL-cholesterol calculated from these data (certified laboratory for clinical chemistry). HbA1c was determined using a Hi-Auto A1C HA-8140 system (Menarini Diagnostics, Germany). Serum insulin was measured using a commercial enzyme-linked immunosorbent assay (Insulin ELISA, Mercodia A B, Uppsala, Sweden). Homeostasis Model Assessment Insulin Resistance (HOMA-IR) was calculated as fasting insulin (IU/L)×fasting glucose (mmol/L)/22.5 (Matthews D R, et al. 1985 Homeostasis model assessment: insulin resistance and beta-cell function from fasting plasma glucose and insulin concentrations in man. Diabetologia 28:412-9).

Human plasma MR-proANP₅₃₋₉₀ was determined as described previously (Morgenthaler N G, et al. 2004 Immunoluminometric assay for the midregion of pro-atrial natriuretic peptide in human plasma. Clin Chem 50:234-6).

NT-proBNP was determined by an electrochemiluminescence immunoassay (ELICIA, Roche Diagnostics, Basel, Switzerland).

### Statistical Analysis

We divided the study population into three groups: patients with essential hypertension (n=54), non hypertensive subjects with central obesity (n=53), and non hypertensive subjects without central obesity (n=55). Central obesity was diagnosed according to the ATP III-defined metabolic syndrome criteria (National Cholesterol Education Program (NCEP) Expert Panel on Detection, Evaluation, and Treatment of High Blood Cholesterol in Adults (Adult Treatment Panel III). Third report of the national Cholesterol Education Program (NCEP) expert panel on detection, evaluation, and treatment of high blood cholesterol in adults (Adult Treatment Panel III). Final report. Circulation 2002; 106:3143-3421).

General characteristics are given as mean ±SD. All other data are presented as means ±SEM. All data were log-transformed before analysis. Delta MR-proANP₅₃₋₉₀ from 0 to 180 min was calculated in oGTT. Group comparison was performed by ANOVA followed by the Sidak test as post hoc multiple group comparison. Repeated measures ANOVA analyses were used for comparison of the time-courses of MR-proANP₅₃₋₉₀ during oGTT between three groups. Correlation analysis was made by Pearson's correlation. In clamp experiments, insulin sensitivity was determined as glucose infusion rate (GIR; mg per kg body weight·min⁻¹) in the steady-state of the clamp test (least 30 min) divided by circulating insulin concentration in steady-state (pmol/l). The nonparametric Wilcoxon's signed-rank test for paired samples was used to compare data from the baseline and in the steady-state. P values <0.05 were considered significant in all analyses. All statistical analyses were performed using SPSS for Windows 14 (SPSS Inc., Chicago, Ill.).

### RESULTS

The characteristics of the study population are summarised in Table 1. Obese normotensive subjects were more insulin resistant compared with non obese normotensive subjects: they had higher fasting insulin and blood glucose concentrations, lower HDL-cholesterol concentration and triglyceride levels, and higher SPB and DBP. Except higher age, HbA1c level and SBP, obese subjects with hypertension were comparable in BMI, waist circumference and insulin resistance with obese normotensive subjects.

The lowest fasting concentrations of MR-proANP₅₃₋₉₀ were observed in obese normotensive subjects, compared with non obese normotensive subjects and obese subjects with hypertension (53.9±28.0 pmol/l vs. 64.1±25.6 pmol/l and 77.5±30.8 pmol/l). After adjustment for age and BMI, the differences remained significant. Fasting MR-proANP₅₃₋₉₀ levels correlated significantly and positively with age (r=0.429, p<0.0001), HDL-cholesterol (r=0.270, p=0.006), and negatively with BMI (r=−0.313, p=0.001), DBP (r=−0.251, p=0.009), fasting insulin (r=−0.276, p=0.004) and HOMA-IR index (r=−0.268, p=0.005) in normotensive subjects. By contrast, in obese subjects with hypertension positive correlations with MR-proANP₅₃₋₉₀ were restricted to age (r=0.546, p<0.0001).

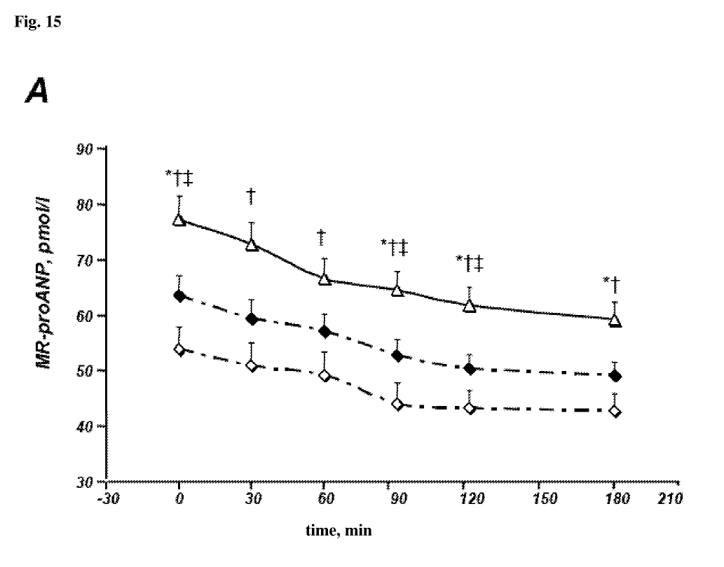

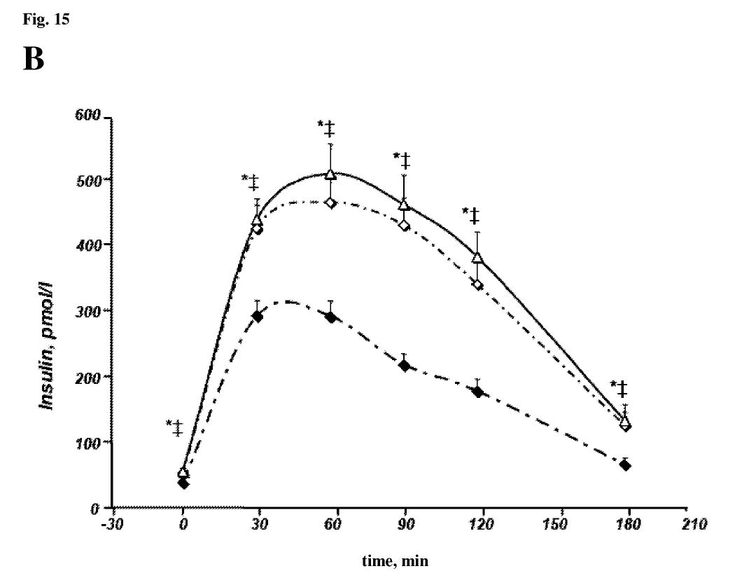

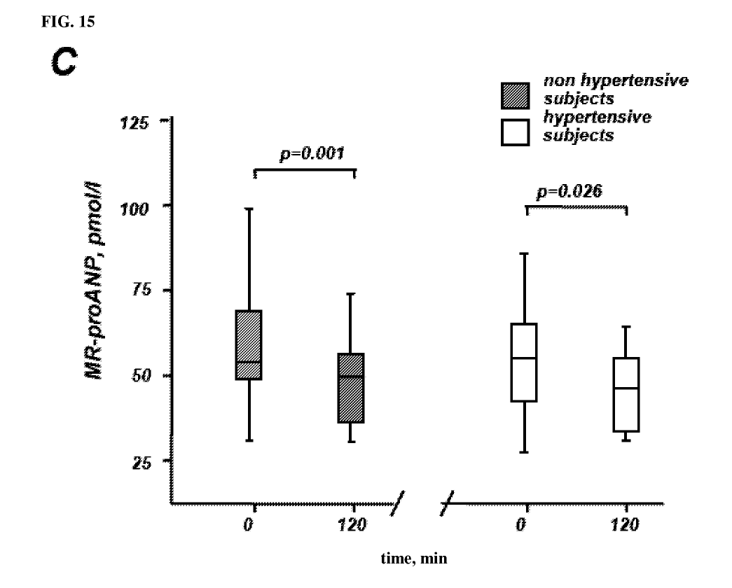

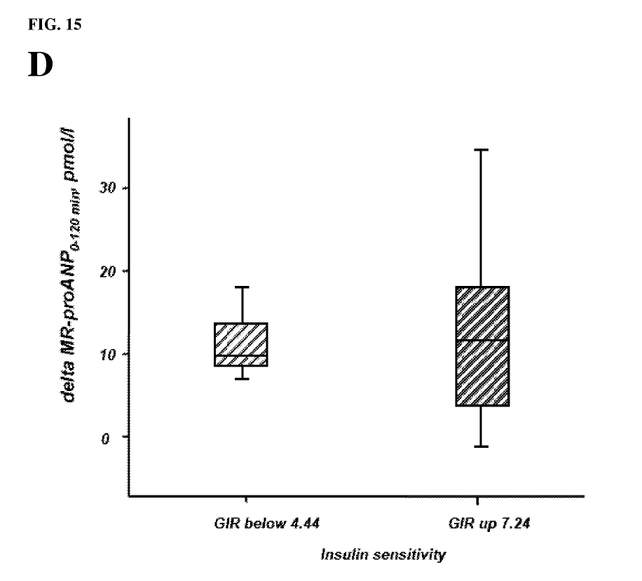

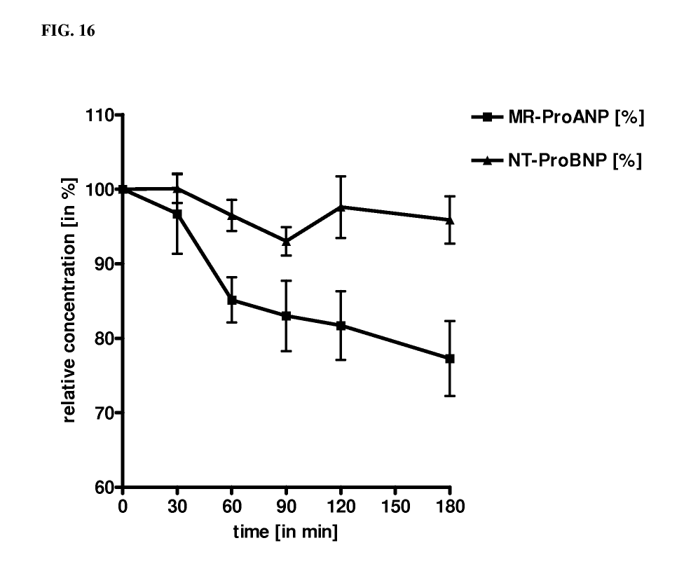

In all study subjects, MR-proANP₅₃₋₉₀ levels decreased rapidly at 30 min after oral glucose challenge and remained suppressed during the entire test (p<0.0001; for basal levels vs. levels at 30, 60, 90, 120 and 180 min of the oGTT). Post challenge concentrations of MR-proANP₅₃₋₉₀ were significantly lower (FIG. 15A) from 90 to 180 min in obese normotensive subjects compared with non obese normotensive subjects and obese hypertensive subjects. The relative suppression of MR-proANP₅₃₋₉₀ at 180 min was 20.0±13.4% in non obese normotensive subjects vs. 21.4±19.5% in obese normotensive subjects vs. 21.2±13.4% in hypertensive subjects (not significant). Fasting and post glucose challenge levels of insulin were significantly lower in obese normotensive subjects (FIG. 15B) and correlated negatively with fasting and post challenge levels of MR-proANP₅₃₋₉₀ in normotensive subjects from 60 to 180 min (r=−0.198-−0.358; p<0.0001-0.05), while these correlations were much weaker in hypertensive subjects. There was no correlation of fasting and post challenge blood glucose levels with MR-proANP₅₃₋₉₀ levels during oGTT in normotensive and hypertensive subjects (data not shown).

Although BNP appears to play a minor role physiologically in healthy subjects, it is usually regulated in a similar manner to ANP. We therefore tested whether NT-proBNP levels also decline in response to oral glucose challenges. NT-proBNP levels were assessed in 10 subjects and compared to MR-proANP₅₃₋₉₀ in this subgroup. Indeed, the relative concentrations of NT-proBNP also declined after the glucose challenge, but the response was more transient and smaller compared to MR-proANP₅₃₋₉₀ (FIG. 16).

Thirty-one obese individuals (17 normotensive subjects and 14 hypertensive subjects) underwent euglycemic hyperinsulinemic clamps. Normotensive subjects were matched for age, BMI, waist circumference and insulin sensitivity with hypertensive subjects (mean ±SE; 30.6±3.5 kg/m2 vs. 32.0±3.4 kg/m2, p=0.341; 101.0±7.3 cm vs. 103.6±8.8 cm, p=0.526; 6.1±1.6 mg/kg body weight×min−1 vs. 5.5±2.0 mg/kg body weight×min−1, p=0.388; respectively). Fasting blood glucose, insulin and MR-proANP₅₃₋₉₀ levels were not different between normotensive and hypertensive subjects (mean ±SE; 5.2±0.5 mmol/l vs. 5.4±0.6 mmol/l; p=0.147; 66.0±40.2 pmol/l vs. 76.2±39.0 pmol/l; p=0.470; 59.5±18.3 pmol/l vs. 61.0±31.5 pmol/l; p=0.874; respectively). In the euglycemic clamp, circulating insulin levels increased to 1223 (range 708-2106) pmol/l in normotensive subjects and to 1247 (range 430-1476) pmol/l in hypertensive subjects at 120 min of the clamp (p=0.489). In both groups MR-proANP₅₃₋₉₀ levels were significantly decreased at 120 min of the clamp compared with basal values, but they did not differ between both groups (A MR-proANP₅₃₋₉₀ 0-120 min 11.1 (−1.3-25.1) pmol/l in normotensive subjects vs. 8.5 (−35.2-34.6) pmol/l in hypertensive subjects; p=0.297) (FIG. 15C). No difference was observed in the suppression of MR-proANP₅₃₋₉₀ in subjects with low and high insulin sensitivity (determined as glucose infusion rate value below the 25^{th} and above the 75^{th} percentile, respectively) (FIG. 15D).

Mean MR-ProADM concentration in healthy individuals (n=264) was 0.33 nmol/L (standard deviation 0.07 nmol/L), range 0.1-0.64 nmol/L, 99^{th} percentile was 0.52 nmol/L, 97.5^{th} percentile was 0.49 nmol/L, 2.5^{th} percentile was 0.17 nmol/L, 1^{st} percentile was 0.14 nmol/L. The lower detection limit of the assay was 0.08 nmol/L (Morgenthaler et al. 2005. Clin Chem 51(10):1823-1829).

Median MR-ProANP₅₃₋₉₀ concentration in healthy individuals (n=325) was 45 pmol/L, range 9.6-313 pmol/L, 99^{th} percentile was 197.5 pmol/L, 97.5^{th} percentile was 163.9 pmol/L, 2.5^{th} percentile was 18.4 pmol/L, 1^{st} percentile was 13.6 pmol/L. The lower detection limit of the assay was 6.0 pmol/L (Morgenthaler et al. 2004. Clin Chem 50(1):234-236).

Median CT-ProAVP concentration in healthy individuals (n=359) was 4.2 pmol/L, range 1-13.8 pmol/L, 99^{th} percentile was 13.5 pmol/L, 97.5^{th} percentile was 11.25 pmol/L, 2.5^{th} percentile was 1.7 pmol/L. The lower detection limit of the assay was 1.7 pmol/L (Morgenthaler et al. 2006. Clin Chem 52(1):112-119). 9 individuals out of 359 had CT-proAVP-values below the lower detection limit and were defined as 1.0 pmol/L.

Mean CT-ProET-1 concentration in healthy individuals (n=326) was 44.3 pmol/L (standard deviation 10.6 pmol/L), range 10.5-77.4 pmol/L, 99^{th} percentile was 72.8 pmol/L, 97.5^{th} percentile was 66.6 pmol/L, 2.5^{th} percentile was 24.8 pmol/L, 1^{st} percentile was 17.4 pmol/L. The lower detection limit of the assay was 0.4 pmol/L (Papassotiriou et al. 2006. Clin Chem 52(6):1144-1151).

Mean NT-proBNP concentration in healthy individuals (n=2264) was 5.94 pmol/l (standard deviation 7.36 pmol/l), the median was 3.25 pmol/l, 97.5^{th} percentile was 19.94 pmol/l and 95^{th} percentile was 17.58 pmol/l. The lower detection limit of the assay was 0.59 pmol/l (Assay proBNP II cobas by Roche; manual 2007-09 V2).

TABLE 1
Clinical and biochemical characteristics of the study subjects

| | Non obese, non | Obese, non | Obese, |
|---|---|---|---|
| | hypertensive | hypertensive | hypertensive |
| | subjects | subjects | subjects |
| | (n = 55) | (n = 53) | (n = 54) |
| Clinical | | | |
| characteristics | | | |
| Age (years) | 47.1 ± 13.7 | 47.9 ± 10.5 | 60.1 ± 8.8 ^{a, d} |
| Sex (female; %) | 74.2 | 73.4 | 66.2 |
| BMI (kg/m²) | 23.4 ± 2.3 | 30.0 ± 5.0 ^{a} | 30.0 ± 4.0 ^{a} |
| Waist circumference | 79.8 ± 8.7 | 100.0 ± 10.4 ^{a} | 100.1 ± 11.4 ^{a} |
| (cm) | | | |
| Systolic blood | 111.3 ± 12.1 | 119.5 ± 13.6 ^{a} | 130.9 ± 16.8 ^{a, d} |
| pressure (mm Hg) | | | |
| Diastolic blood | 70.6 ± 8.2 | 78.5 ± 8.3^{a} | 81.6 ± 10.9 ^{a} |
| pressure (mm Hg) | | | |
| Biochemical | | | |
| characteristics | | | |
| Fasting blood glucose | 4.7 ± 0.4 | 5.0 ± 0.5 ^{b} | 5.0 ± 0.6 ^{b} |
| (mmol/l) | | | |
| HbAlc (%) | 5.3 ± 0.4 | 5.3 ± 0.4 | 5.6 ± 0.4 ^{a, d} |
| HDL cholesterol | 1.5 ± 0.3 | 1.3 ± 0.3 ^{a} | 1.3 ± 0.3 ^{a} |
| (mmol/l) | | | |
| Triglycerides | 1.0 ± 0.6 | 1.6 ± 0.9 ^{b} | 1.6 ± 0.8 ^{a} |
| (mmol/l) | | | |
| Fasting insulin | 36.8 ± 47.0 | 52.9 ± 34.9 ^{a} | 54.1 ± 35.8 ^{a} |
| (pmol/l) | | | |
| HOMA_{IR} | 1.3 ± 1.9 | 2.0 ± 1.4 ^{a} | 2.0 ± 1.5 ^{a} |
| (mU mmol/l) | | | |
| proANP (pmol/l) | 64.1 ± 25.6 | 53.9 ± 28.0 ^{c} | 77.5 ± 30.8 ^{d} |
| 0 min | | | |
| delta proANP_{(0-180 min)} | 14.7 ± 11.9 | 10.9 ± 11.0 ^{c} | 18.2 ± 15.3 ^{d} |
| (pmol/l) | | | |

Unless otherwise indicated, values are means ± SD. All values are unadjusted.
^{a} p < 0.0001;
^{b} p < 0.01;
^{c} p < 0.05 vs. non obese, non hypertensive subjects and
^{d} p < 0.0001;
^{e} p < 0.05 vs.obese non hypertensive subjects.
Obesity was defined as “central obesity” by ATIII Criteria for metabolic syndrome: waist circumference for women >88 cm and >102 cm for men.

1. An in vitro-method for classifying a foodstuff and/or beverage and/or diet and/or nutrition regimen and/or medicament in view of an effect on the cardiovascular system of a subject, comprising determining the relative level of one or more cardiovascular markers in said subject.

2. The in vitro-method according to claim 1, wherein the postprandial relative level of one or more cardiovascular markers is determined.

3. The in vitro-method according to claim 1, wherein the relative level of one or more cardiovascular markers is determined with an assay having a sensitivity 1 nmol/L or lower, preferably 100 pmol/L or lower, more preferably 10 pmol/L or lower, even more preferably 1 pmol/L or lower, most preferably 0.5 pmol/L or lower.

4. The in vitro-method according to claim 1, further comprising:
a. determining the basal level of one or more cardiovascular markers in said subject,
b. determining the postprandial level of said one or more cardiovascular markers,
c. calculating the relative level of one or more cardiovascular markers from the values obtained in steps a and b.

5. The in vitro-method according to claim 1, wherein a postprandial relative increase of the level of said one or more cardiovascular markers of more than 5%, preferably between more than 5 and up to 20% in said subject is correlated with a positive effect of said foodstuff and/or beverage and/or diet and/or nutrition regimen and/or medicament on the on the cardiovascular system of said subject, wherein a postprandial relative increase or decrease of the level of said one or more cardiovascular markers of about 5% in said subject is correlated to a neutral effect of said foodstuff and/or beverage and/or diet and/or nutrition regimen and/or medicament on the cardiovascular system of said subject, and wherein a postprandial relative decrease of the level of said one or more cardiovascular markers of more than 5% in said subject is correlated to a negative effect of said foodstuff and/or beverage and/or diet and/or nutrition regimen and/or medicament on the cardiovascular system of said subject.

6. The in vitro-method according to claim 4, wherein the basal level of said cardiovascular marker and the postprandial level of said cardiovascular marker in said subject are determined with an immunoassay.

7. The in vitro-method according to claim 1, wherein one of the cardiovascular markers is proANP or fragments thereof, more preferred MR-proANP, most preferred MR-proANP₅₃₋₉₀.

8. A method for determining the change of the level of one or more cardiovascular markers of a subject relative to the basal level of said markers of said subject, comprising performing an assay capable of detecting a decrease of the level of said one or more cardiovascular markers and capable of detecting an increase of the level of said one or more cardiovascular markers.

9. The method according to claim 8, wherein the change is an increase or a decrease, and wherein the assay has sensitivity of 1 nmol/L or lower.

10. The method according to claim 8 for determining the postprandial change of the level of one or more cardiovascular markers of a subject relative to the basal level of said markers of said subject.

11. The method according to claim 8, wherein the assay is an immunoassay.

12. The method according to claim 8, wherein said change in the level of one or more cardiovascular markers is used to classify a foodstuff and/or beverage and/or diet and/or nutrition regimen and/or medicament in view of an effect on the cardiovascular system of a subject.

13. The method according to claim 8, wherein said change in the level of one or more cardiovascular markers is used to classify a diabetes medicament.

14. The method according to claim 8, wherein said subject is a human being having a medical condition associated with the cardiovascular system and/or metabolic system

15. The method according to claim 8, wherein said subject is a human being suffering from diabetes.

16. The method according to claim 8, wherein at least one of the markers is a peptide selected from the group comprising natriuretic peptides, endothelin-1, vasopressin, adrenomedullin, as well as propeptides thereof and fragments of at least 3 amino acids thereof.

17. The in vitro-method of claim 1, wherein said change in the level of one or more cardiovascular markers is used to classify a diabetes medicament.

18. The in vitro-method of claim 1, wherein said subject is a human being having a medical condition associated with the cardiovascular system and/or metabolic system

19. The in vitro-method of claim 1, wherein said subject is a human being suffering from diabetes.

20. The in vitro-method of claim 1, wherein at least one of the markers is a peptide selected from the group comprising natriuretic peptides, endothelin-1, vasopressin, adrenomedullin, as well as propeptides thereof and fragments of at least 3 amino acids thereof.


SEQUENCE LISTING (14 sequences)

SEQ ID NO: 1 - protein, 126 aa, Homo sapiens
NPMYNAVSNADLMDFKNLLDHLEEKMPLEDEVVPPQVLSEPNEEAGAALSPLPEVPPWTGEVSPAQRDGGALGRGPWDSSDRSALLKSKLRALLTAPRSLRRSSCFGGRMDRIGAQSGLGCNSFRY

SEQ ID NO: 2 - protein, 185 aa, Homo sapiens
MKLVSVALMYLGSLAFLGADTARLDVASEFRKKWNKWALSRGKRELRMSSSYPTGLADVKAGPAQTLIRPQDMKGASRSPEDSSPDAARIRVKRYRQSMNNFQGLRSFGCRFGTCTVQKLAHQIYQFTDKDKDNVAPRSKISPQGYGRRRRRSLPEAGPGRTLVSSKPQAHGAPAPPSGSAPHFL

SEQ ID NO: 3 - protein, 164 aa, Homo sapiens
ARLDVASEFRKKWNKWALSRGKRELRMSSSYPTGLADVKAGPAQTLIRPQDMKGASRSPEDSSPDAARIRVKRYRQSMNNFQGLRSFGCRFGTCTVQKLAHQIYQFTDKDKDNVAPRSKISPQGYGRRRRRSLPEAGPGRTLVSSKPQAHGAPAPPSGSAPHFL

SEQ ID NO: 4 - protein, 40 aa, Homo sapiens
ELRMSSSYPTGLADVKAGPAQTLIRPQDMKGASRSPEDSS

SEQ ID NO: 5 - protein, 52 aa, Homo sapiens
YRQSMNNFQGLRSFGCRFGTCTVQKLAHQIYQFTDKDKDNVAPRSKISPQGY

SEQ ID NO: 6 - protein, 212 aa, Homo sapiens
MDYLLMIFSLLFVACQGAPETAVLGAELSAVGENGGEKPTPSPPWRLRRSKRCSCSSLMDKECVYFCHLDIIWVNTPEHVVPYGLGSPRSKRALENLLPTKATDRENRCQCASQKDKKCWNFCQAGKELRAEDIMEKDWNNHKKGKDCSKLGKKCIYQQLVRGRKIRRSSEEHLRQTRSETMRNSVKSSFHDPKLKGKPSRERYVTHNRAHW

SEQ ID NO: 7 - protein, 195 aa, Homo sapiens
APETAVLGAELSAVGENGGEKPTPSPPWRLRRSKRCSCSSLMDKECVYFCHLDIIWVNTPEHVVPYGLGSPRSKRALENLLPTKATDRENRCQCASQKDKKCWNFCQAGKELRAEDIMEKDWNNHKKGKDCSKLGKKCIYQQLVRGRKIRRSSEEHLRQTRSETMRNSVKSSFHDPKLKGKPSRERYVTHNRAHW

SEQ ID NO: 8 - protein, 21 aa, Homo sapiens
CSCSSLMDKECVYFCHLDIIW

SEQ ID NO: 9 - protein, 45 aa, Homo sapiens
RSSEEHLRQTRSETMRNSVKSSFHDPKLKGKPSRERYVTHNRAHW

SEQ ID NO: 10 - protein, 38 aa, Homo sapiens
CSCSSLMDKECVYFCHLDIIWVNTPEHVVPYGLGSPRS

SEQ ID NO: 11 - protein, 164 aa, Homo sapiens
MPDTMLPACFLGLLAFSSACYFQNCPRGGKRAMSDLELRQCLPCGPGGKGRCFGPSICCADELGCFVGTAEALRCQEENYLPSPCQSGQKACGSGGRCAAFGVCCNDESCVTEPECREGFHRRARASDRSNATQLDGPAGALLLRLVQLAGAPEPFEPAQPDAY

SEQ ID NO: 12 - protein, 145 aa, Homo sapiens
CYFQNCPRGGKRAMSDLELRQCLPCGPGGKGRCFGPSICCADELGCFVGTAEALRCQEENYLPSPCQSGQKACGSGGRCAAFGVCCNDESCVTEPECREGFHRRARASDRSNATQLDGPAGALLLRLVQLAGAPEPFEPAQPDAY

SEQ ID NO: 13 - protein, 9 aa, Homo sapiens
CYFQNCPRG

SEQ ID NO: 14 - protein, 39 aa, Homo sapiens
ASDRSNATQLDGPAGALLLRLVQLAGAPEPFEPAQPDAY